(19) United States
(12) Patent Application Publication
(10) Pub. No.: US 2003/0125658 A1
(43) Pub. Date: Jul. 3, 2003

(54) Tampon applicator assembly and method of using same

(76) Inventor: Jennifer Marvin, Greenville, WI (US)

Correspondence Address: Andrew D Stover, Brinks Hofer Gilson & Lione, PO BOX 10395, Chicago, IL, 60610, US

(21) Appl. No.: 10/038,765

(22) Filed: Jan. 2, 2002

(51) Int. Cl. A61F 13/20

(52) U.S. Cl. 604/13; 604/385.18; 604/904

(57) ABSTRACT

A tampon applicator assembly and a method for placing a tampon within a vagina. The assembly includes an expandable tampon and a single piece applicator. The applicator assists in the proper placement of the tampon into the vagina. Once inside the vagina, the tampon expands to its expanded position, and absorbs body fluid.

### FIELD OF THE INVENTION

The present invention relates to a catamenial tampon applicator assembly and a method of using the assembly. More specifically, the invention relates to an applicator assembly which includes a single piece tampon applicator having a tampon secured at its leading end.

### BACKGROUND

Conventional tampon applicators typically are made of two pieces. The first piece is an outer housing or carriage for holding the tampon, and the second piece is an actuator or plunger. In use, the applicator is inserted into the vagina, and the plunger is pushed by the user to expel the tampon from the housing.

While the conventional two-piece applicator has been an effective and efficient means of inserting a tampon, it has a few limitations. First, the plunger adds length to the applicator. Second, the leading end of the applicator typically is made of a rigid or semi-rigid material, which can cause discomfort during insertion.

Accordingly, there remains a need in the art for an improved tampon applicator that may be made of a smaller size and that is more comfortable to the user.

### BRIEF SUMMARY

In one aspect of the invention, an assembly is provided for inserting a tampon. The assembly includes a tampon, a withdrawal member, and a single piece applicator. The tampon is adapted to be placed in a vagina to absorb body fluid. The tampon has a first end portion and a second end portion. The withdrawal member is attached to at least one of the first end portion and the second end portion of the tampon, and the withdrawal member includes a stopping member. The single piece applicator has a leading end portion and a trailing end portion, and the leading end portion engages the tampon. The trailing end portion is adapted to releasably secure the stopping member against movement in at least one direction.

In another aspect of the invention, an assembly includes a single piece applicator having a trailing end portion and a leading end portion, and an expandable tampon having an expanded position and a contracted position. The tampon is adapted to be placed in a vagina to absorb body fluid. The leading end portion of the applicator engages the tampon while the tampon is in its contracted position.

In yet another aspect of the invention, a method of placing a tampon within a vagina is provided. The method includes inserting a tampon applicator assembly into a vagina, the assembly including a single piece applicator and an expandable tampon. The tampon is released from the single piece applicator. The single piece applicator is then withdrawn from the vagina.

Other aspects of the invention will be apparent to those skilled in the art in view of the claims following the detailed description of the presently preferred embodiments.

### BRIEF DESCRIPTION OF THE DRAWINGS

FIG. 1A is a perspective view of one embodiment of a tampon applicator assembly according to the present invention.

FIG. 1B is a cross-sectional view of the assembly of FIG. 1A.

FIGS. 2A-2C are perspective views of alternate embodiments of the assembly of FIGS. 1A-1B.

FIG. 3 is a side view of one embodiment of a tampon used with the assembly of the present invention.

FIG. 4 is a perspective view of another embodiment of a tampon used with the assembly of the present invention.

FIG. 5A is a side view of an alternate embodiment of a tampon used with the assembly of the present invention.

FIG. 5B is a top view of the tampon of FIG. 5A.

FIG. 5C is a perspective view of the tampon of FIGS. 5A-5B.

FIG. 6A is a perspective view of an alternate embodiment of an assembly according to the present invention.

FIG. 6B is a cross-sectional view of the assembly of FIG. 6A.

FIG. 7A is a side view of an alternate embodiment of an assembly according to the present invention.

FIG. 7B is a cross-sectional view of the assembly of FIG. 7A.

### DETAILED DESCRIPTION

The present invention provides an easy to use, comfortable, and effective assembly for inserting a catamenial tampon into a vagina. The assembly according to the present invention includes a tampon and a single piece applicator. The single piece applicator cooperates with the tampon during insertion of the assembly into the vagina. The tampon is the leading edge of the assembly, and thus provides a softer, more comfortable insertion than would a leading end of some other applicators. After the tampon is properly positioned within the body, the tampon and the single piece applicator are separated, and the applicator is withdrawn while the tampon is left in the body. The single piece applicator does not include a plunger, and thus may be more economical to make and may be made of smaller dimensions.

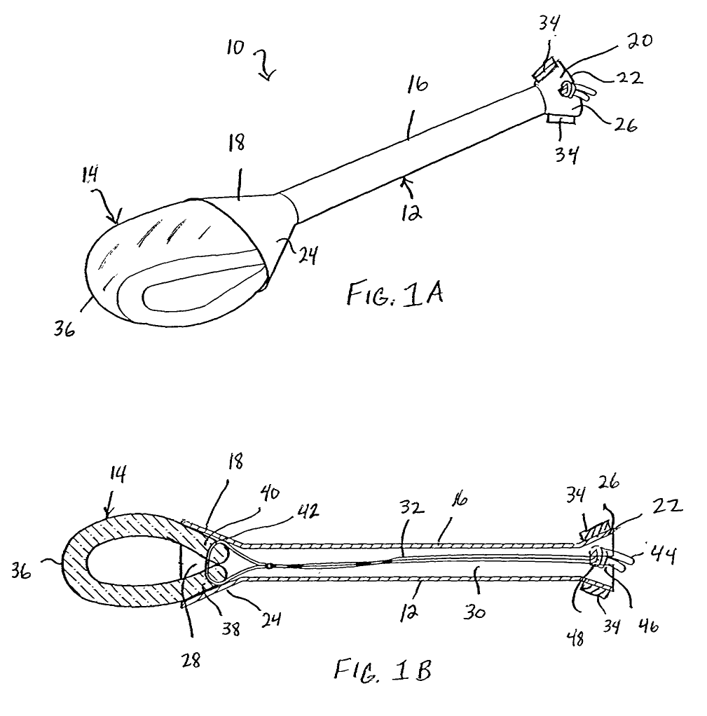

Referring now to FIGS. 1A and 1B, one embodiment of a tampon assembly in accordance with the present invention is shown generally at 10. The assembly 10 includes a single piece applicator 12 and a tampon 14. The applicator 12 includes an elongated portion 16, a leading end portion 18, and a trailing end portion 20, which has a trailing edge 22. In the embodiment shown in FIGS. 1A and 1B, the leading end portion 18 and the trailing end portion 20 have generally conical shapes 24, 26, respectively. The conical shape 24 of the leading end portion 18 defines a cavity 28. The applicator 12 has a hollow interior or passageway 30 which allows a withdrawal member 32 to extend therethrough from the leading end portion 18 to the trailing end portion 20. The withdrawal member 32 allows the user to withdraw the tampon 14 at a time after it has been inserted into the body. The trailing end portion 20 preferably includes at least one orientation indicator 34, such as one or more tabs, which assist the user in properly orientating the applicator, and thus the tampon, within the vagina, as described in more detail below.

The applicators of the present invention should be made of a biocompatible material, such as a paperboard stock, or a plastic. In a preferred embodiment, the applicators have an elliptical cross-section, which maximizes the space available to engage the tampon 14, and more closely matches the cross-section of the vagina.

The withdrawal member 32 can be constructed from various types of strings, threads or ribbons. A thread or ribbon made from 100 percent cotton fibers works well. The withdrawal member 32 can be dyed and/or treated with an anti-wicking agent, such as wax, before being secured to its respective tampon 14. The anti-wicking agent will reduce body fluids from wicking along the withdrawal member 32. In other embodiments, the withdrawal member 32 may be formed from a thermoplastic, such as polypropylene or nylon.

The tampon 14 is preferably made of a generally absorbent and soft material. A “tampon” as used herein refers to absorbent specifically designed, configured, and/or adapted for placement into a vagina in order to absorb body fluid during the menstrual cycle. More particularly, the tampon 14 is designed and configured to be placed above the introital region of a woman's vagina so as to absorb and intercept the flow of menses, blood, and other body fluids.

Although the assembly 10 may use any tampon known in the art, in the preferred embodiment shown, the tampon 14 is designed to be expandable, that is, it is designed to expand from a contracted position into an expanded position. The expansion may be accomplished by making at least a portion of the tampon 14 from an expandable resilient material. The expansion of the tampon 14 while in use significantly reduces leakage of body fluid from a woman's vagina, especially during the peak of her menstrual cycle.

The assembly 10 of the present invention is constructed and arranged such that the tampon 14 engages the leading end portion of the applicator when the tampon 14 is in its contracted position. In the embodiment shown in FIGS. 1A and 1B, the tampon 14 includes a leading portion 36 and first and second trailing end portions 38, 40, respectively. The leading end portion 18 of the applicator 12 is adapted to engage the tampon 14 in its contracted position in the cavity 28 which houses and holds the end portions 38, 40 of the tampon 14 against the tampon's tendency for outward expansion.

The withdrawal member 32 has a first end portion 42 adapted for attachment to the tampon 14 and an opposite, trailing end portion 44. The end portion 42 of the withdrawal member is attached to one or both of the end portions 38, 40 of the tampon 14, and the other end portion 44 of the withdrawal member 32 is releasibly secured to the applicator 12. The withdrawal member 32 is kept at least slightly tensioned such that end portions 38, 40 of the tampon 14 do not disengage from the cavity 28 of the leading end portion 18 of the applicator 12 during insertion of the assembly 10.

The trailing end portion 20 has been adapted to releasably secure the withdrawal member 32. The trailing end portion 20 has a channel or aperture 46 extending from a trailing edge 22 of the applicator 12 and into the surface of a conical shape 26 which defines the surface of the trailing end portion 20 of the applicator 12. The channel or aperture 46 is sized to accommodate the withdrawal member 32, but is adapted to prevent movement of the withdrawal member in one or more directions, such as towards the leading end portion 18 of the applicator 12.

The orientation indicators 34 may be used to facilitate insertion of the assembly 10, and then to properly orientate the tampon 14 inside the vagina so as to maximize its effectiveness. The opening to the vagina is long and narrow. The narrowest part of the leading end portion 36 of the tampon 14, and the narrowest part of the elliptical shaped leading end portion 18 of the applicator 12, should be matched with the narrow dimension of the vagina. In the embodiment shown in FIGS. 1A and 1B, such an alignment would require the assembly to be rotated so that orientation indicators 34 are about 90 degrees from the position in which they are shown. The orientation indicators 34 also provide an approximation of the orientation of the tampon 14 within the vagina in order to guide the user in proper orientation once the tampon 14 is inserted.

After proper orientation of the tampon 14, the withdrawal member 32 is released from the applicator 12 by removing the withdrawal member 32 from the aperture 46. The applicator 12 is then removed from the vagina, while the tampon 14 remains inside. In some embodiments of the assembly, a slight rotation or twisting of the applicator may be necessary to dislodge the tampon. The release of the withdrawal member 32, and thus the release of the tension on the end portions 38, 40 of the tampon 14, allows the tampon 14 to disengage from the leading end portion 18 of the applicator 12. The withdrawal member 32 slides through the passageway 30 as the applicator 12 is removed from the body, and the tampon 14 opens to its expanded position within the body.

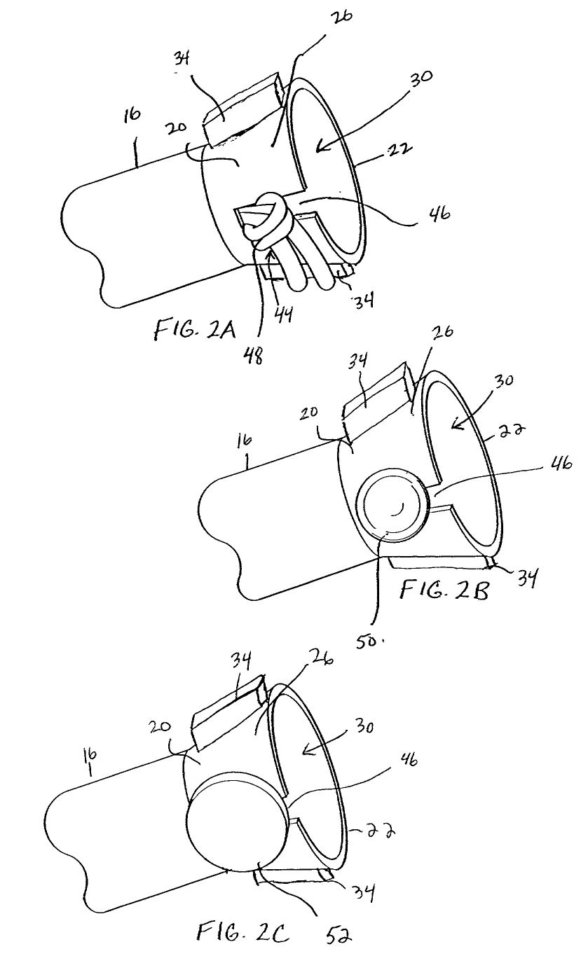

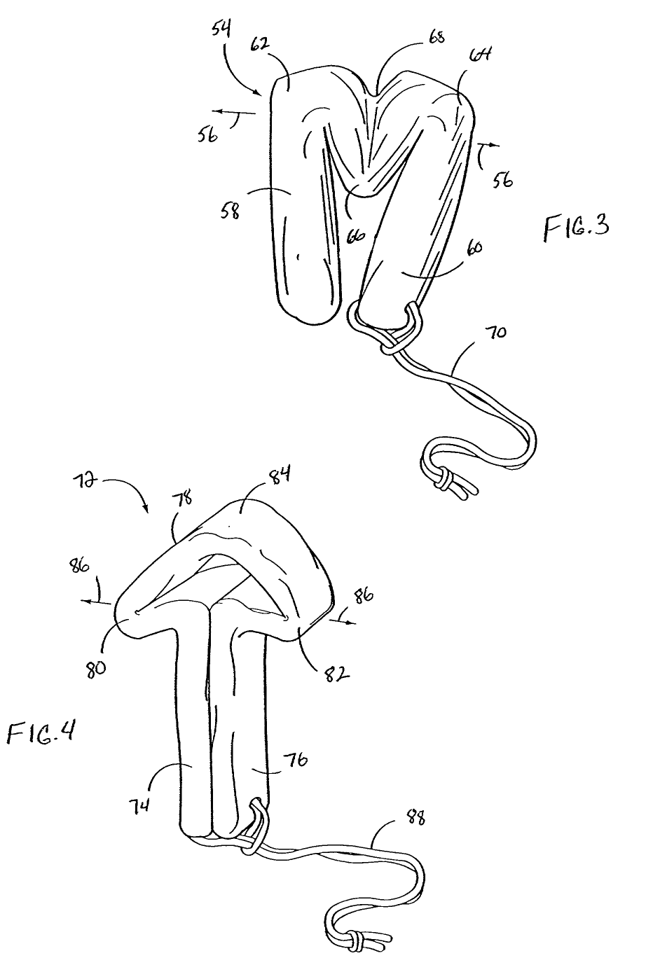

FIGS. 2A-2C illustrate particularly preferred embodiments of the trailing end portion 20 of the applicator 12 and the end portion 44 of the withdrawal member 32. In the embodiments shown, the aperture 46 is sized to prevent movement of a stopping member at the trailing end portion 44 of the withdrawal member 32, in at least the direction towards the leading end portion of the applicator 12. After the insertion of the tampon applicator assembly 10 into the body, the user manually pulls the stopping member and the trailing end portion 44 of the withdrawal member 32 out of the aperture 46 so that it can move freely through passageway 30, thus allowing separation of the tampon and the single piece applicator 12, and removal of the applicator 12 from the body, while leaving the tampon 14 within the body.

In FIG. 2A, the stopping member is a knot 48. The knot 48 also serves to prevent fraying of the withdrawal member 32 and to provide a place or point where a woman can grasp the withdrawal member 32 when she is ready to remove the tampon 14 from her vagina.

FIG. 2B illustrates an alternate embodiment of a stopping member. The stopping member is formed as a bead 50, preferably made by melting the end of the withdrawal member 32. In this embodiment, the withdrawal member 32 is preferably made from a thermoplastic material, such as polypropylene or nylon, that will readily form a bead upon melting. FIG. 2C illustrates another embodiment of a stopping member which is a separate element, such as a disk 52, attached to the end of the withdrawal member 32.

FIGS. 3-5 illustrate various expandable tampons that may be used in accordance with the present invention. FIG. 3 illustrates a tampon 54 having a “M-shape” profile in a plane parallel to the directions indicated by arrows 56. Such tampons are more fully disclosed in commonly assigned U.S. Pat. No. 6,039,828, entitled “Method Of Forming A Tampon Having A Resilient Member”; U.S. Pat. No. 6,039,716, entitled “Laterally Expandable Tampon”; U.S. Pat. No. 5,795,346, entitled “Tampon Having A Resilient Member”; U.S. Pat. No. 5,755,906, entitled “Method Of Forming A Tampon Having A Resilient Member”, and U.S. Pat. No. 5,659,934, entitled “Method Of Forming A Laterally Expandable Tampon”, all of which are hereby incorporated by reference in their entireties. Briefly, the tampon 54 is formed from an elongated member having a first end portion 58 and a second end portion 60, and has three folds, 62, 64, and 66. The first end portion 58 and the second end portion 60 are aligned generally adjacent to each other and, together with the folds 62, 64, 66, form a generally planar M-shaped profile. An inward crease 68 is formed between the fold 62 and the fold 64, and opposite the fold 66. One or both of the end portions are attached to a withdrawal member, such as a string 70. The tampon 54 preferably includes a resilient material or layer (not shown) that functions to expand the tampon 54 in at least a direction indicated by arrows 56 once the tampon 54 is placed within the vagina.

FIG. 4 illustrates an alternate embodiment of a tampon 72 which is similar in construction to the tampon 54 of FIG. 3, but has a generally “domeshaped” profile. Briefly, the tampon 72 is formed from an elongated member that includes a first end portion 74 and a second end portion 76, and a connecting portion 78. The first end portion 74 and the second end portion 76 are aligned adjacent to each other, and the connecting portion 78 includes three folds 80, 82, 84 in order to form a generally planar closed-loop, domeshaped profile. The dome-shaped profile is in a plane parallel to the direction indicated by arrows 86. In this embodiment, both end portions 74, 76 are attached to a withdrawal member 88. The tampon 72 preferably includes a resilient material or layer (not shown) that functions to expand the tampon 72 in at least a direction indicated by arrows 86 once the tampon 72 is placed within the vagina.

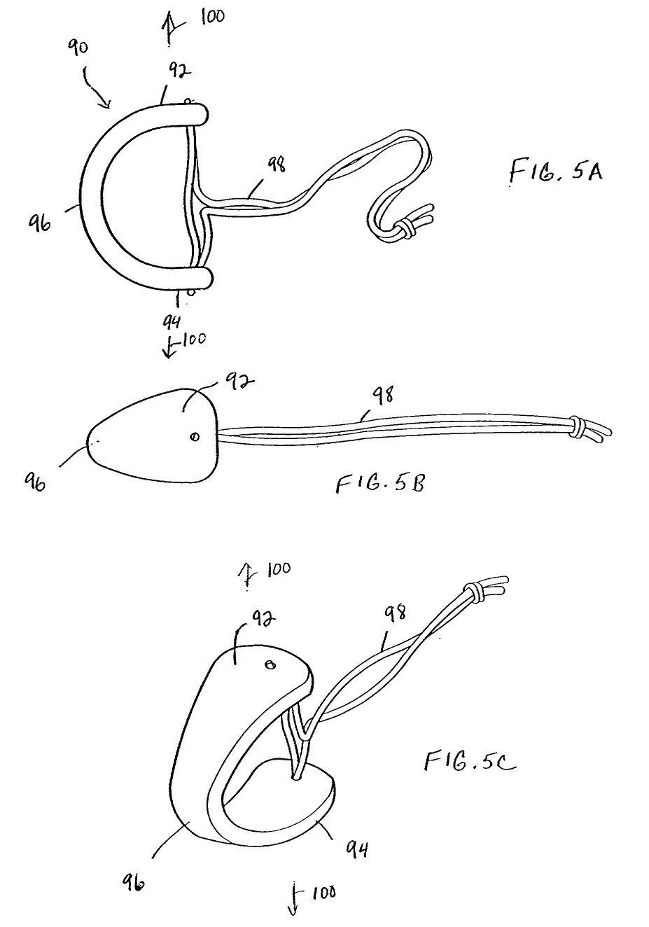

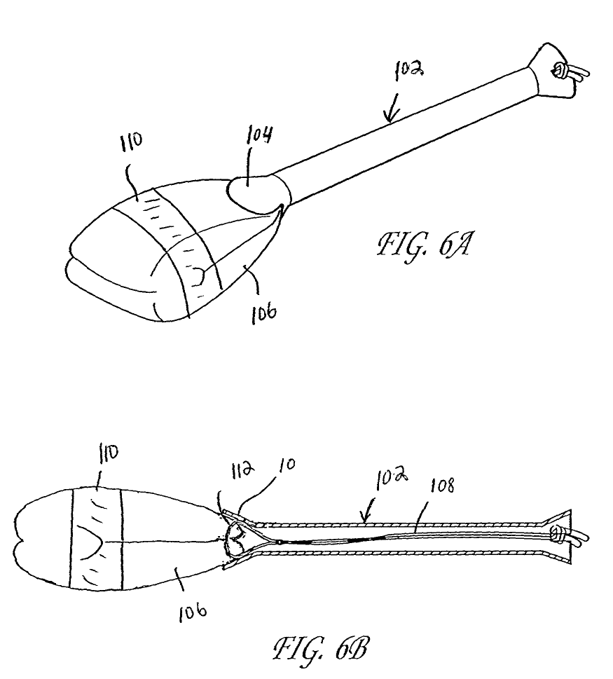

FIGS. 5A-5C illustrate another embodiment of a tampon in accordance with the present invention. The tampon 90 includes a first end portion 92, a second end portion 94, and a connecting portion 96. A withdrawal member 98 is preferably attached at one or both of the first and second end portions 92, 94. The portions 92, 94, and 96 are integrally joined and form a generally C-shaped profile (in a plane parallel to the paper in FIG. 5A) when the tampon 90 is in its expanded position. As shown in FIGS. 5B and 5C, the tampon 90 is tapered such that the first and second end portions 92, 94 are wider than the connecting portion 96 in a direction generally normal to a plane defined by the C-shaped profile. Like the tampons of FIGS. 3 and 4, the tampon 90 preferably includes a resilient material or layer (not shown) that functions to expand the tampon 90 in at least a direction indicated by arrows 100 once the tampon 90 is placed within the vagina.

As stated above, the tampons preferably include a resilient material. The resilient material should be capable of having what is known as “dry and wet expansion” characteristics. In other words, the resilient material should be made of a material which is capable of expanding back to or towards its original configuration in either a dry state, a wet state, and preferably, in both a dry and a wet state. Dry expansion of the resilient material is beneficial in that the tampon does not have to be wetted by body fluid before the resilient material is capable of expanding. Furthermore, it is advantageous to employ a material for the resilient material which is capable of expanding even while the tampon is wetted by body fluid. For purposes of this invention, the resilient material can be a resilient foam, such as a closed cell foam or an open cell foam. Other materials from which the resilient material can be made from include polyethylene oxide (PEO) and polyvinyl alcohol (PVA). The resilient material can also be formed from resilient fibers such as polyolefin based fibers, polyethylene oxide fibers, hydrophobic rayon fibers and the like which preferably will have characteristics similar to those of a resilient foam. The resilient material can also be made or constructed from a wettable foam. A polyethylene closed cell foam having good flexibility characteristics also works well.

The absorbent material of the tampon is preferably constructed from a absorbent sheet or ribbon including a blend of cotton and rayon fibers. Two processes for forming such an absorbent sheet are known as “carding” and “airlaying.” Depending upon the desired absorbency one desires in the finished tampon, the basis weight of the absorbent sheet can vary. The U.S. Food and Drug Administration (FDA) has set absorbency standards for “junior”, “regular”, “super” and “super-plus” size tampons. In order to meet the FDA standards for these four sizes, the absorbent sheets are targeted to have basis weights of about 100 grams per square meter (gsm), 120 gsm, 170 gsm and 230 gsm, respectively. Typically, the carding process is controlled to produce an absorbent sheet with a width of between about 40 mm to about 60 mm, preferably about 50 mm. The basis weight and/or the length of the absorbent can also be adjusted to form the different size tampons.

Alternatively, the absorbent material can be formed from absorbent fibers which are assembled and compressed into a generally elongated and/or cylindrical configuration. The absorbent material is preferably formed from cellulosic fibers, such as cotton and rayon. The absorbent can be 100% cotton, 100% rayon, or a blend of cotton and rayon fibers. Some blends which have been tried and work well include a blend of about 15% cotton to about 85% rayon; about 70% cotton to about 30% rayon; about 60% cotton to about 40% rayon; about 25% cotton to about 75% rayon; and about 6% cotton to about 94% rayon. The particular blend of fibers can vary depending upon one's preference.

When cotton fibers are used, the cotton fibers should have a staple length of between about 5 millimeters (mm) to about 20 mm. The cotton fibers should generally have a fiber size of between about 150 microns to about 280 microns. The cotton fibers can also be bleached if desired. Bleaching will make the cotton fibers whiter in appearance.

When rayon fibers are present, the rayon fibers should have a staple length of between about 20 mm to about 35 mm. The rayon fibers should have a denier of between about 2 to about 6. Denier is a unit of fineness of yarn based on a standard of 50 milligrams (mg) for 450 meters of yarn. The rayon fibers can have a circular, a bi-lobal, a tri-lobal cross-sectional configuration, or some other cross-sectional configuration known to those skilled in the art. The bi-lobal configuration has a cross-sectional profile which looks like a dog bone while the tri-lobal configuration has a cross-sectional profile which looks like a “Y”. The rayon fibers can also be bleached if desired.

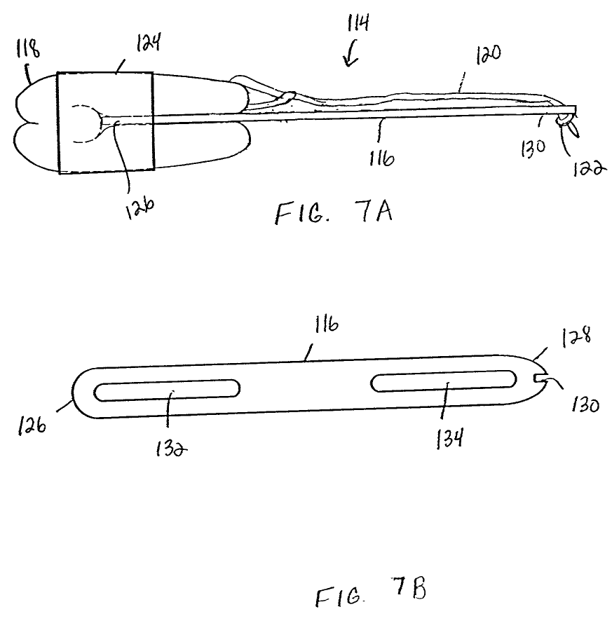

FIGS. 6A-6B illustrate an alternate embodiment of an assembly according to the present invention. As shown in FIG. 6A, applicator 102 includes a leading end portion 104 that is clam-shaped, thereby further facilitating separation of the applicator 102 from the tampon 106 by slight rotation of the applicator after it has been inserted and the withdrawal member 108 has been released. Furthermore, a dissolvable band 110 has been added to the surface of the tampon 106. The dissolvable band 110 holds the tampon 106 against expansion prior to use, but dissolves when placed in contact with moisture inside the body. In the preferred embodiment, the dissolvable band is made from a starch-based film with a pH of about 3.5 to about 6.5, which is generally of the same magnitude of the pH typically found in the vagina. Suitable materials for the dissolvable band 110 include lypholized mucin; a mucopolysacharride polymer matrix; polymers such as xanthan gum or hydroxypropyl cellulose, which are stabilized with antimicrobials and anti-bioadhesion chemicals such as zosteric acid; and anhydrous systems such as a neutral triglyceride wax. One example of a suitable material is Softisan 378, sold by Sasol North America, Inc. (formerly the Condea Vista Company), having an office at 900 Threadneedle, Suite 100, Houston, Tex. 77079.

In the embodiment shown, the dissolvable band 110 is used in addition to a cavity 112 and a withdrawal member 108 for holding the tampon 106; however, in alternate embodiments, the dissolvable band 110 may be used in place of one or both of the cavity 112 and the withdrawal member 108. Moreover, while in the embodiment shown, the dissolvable band 110 covers only a portion of the outer surface of the tampon 106, in alternate embodiments, the band 110 may cover the entire surface of the tampon 106. Furthermore, in another alternate embodiment, a dissolvable coating is used around at least a portion of the tampon.

In an alternate embodiment of the present invention, shown in FIGS. 7A and 7B, a tampon applicator assembly 114 includes an elongated, generally flat and planar single piece applicator 116, a tampon 118, a withdrawal member 120 having a stopping member 122, and a dissolvable band 124. Although the stopping member 122 shown in FIGS. 7A and 7B is formed as a knot, those skilled in the art will appreciate that the stopping member could be formed as a bead, a washer, a disk, or any other suitable shape. The applicator 116 has a leading end portion 126 and a trailing end portion 128 having an aperture 130. The aperture 130 accommodates the withdrawal member 120 but prevents passage of the stopping member 122 and thus prevents movement of the withdrawal member 120 in at least one direction, such as generally towards the leading end portion 126. The leading end portion 126 of the applicator 116 has no features that secure or hold the tampon 118; rather, the dissolvable band 124 secures the tampon 118 in its contracted position, and the withdrawal member 120, when at least slightly tensioned, cooperates to hold the tampon 118 in engagement with the applicator 116. The assembly preferably includes cut-out portions 132, 134, which help limit the overall weight of the assembly, as well as provide a gripping portion for the user. After insertion, body moisture dissolves the band 124, and the user releases the stopping member 122 from the aperture 130. As the tampon 118 opens to its expanded state, the tampon 118 is separated from the applicator 116, which is removed from the body.

In accordance with another aspect of the present invention, a method is provided for placing a tampon within a vagina. The method includes (1) inserting a tampon applicator assembly having a tampon and a single piece applicator into a vagina, (2) releasing the tampon from the single piece applicator, and (3) removing the single piece applicator from the vagina. Depending upon the embodiment of the assembly used in the method, the releasing of the tampon from the single piece applicator may entail either releasing the withdrawal member from the applicator, or dissolving a coating or band placed around the tampon, or twisting of the applicator, or any combination of the above.

Although the invention has been described and illustrated with reference to specific illustrative embodiments thereof, it is not intended that the invention be limited to those illustrative embodiments. Those skilled in the art will recognize that variations and modifications can be made without departing from the true scope and spirit of the invention as defined by the claims that follow. It is therefore intended to include within the invention all such variations and modifications as fall within the scope of the appended claims and equivalents thereof.

1. A tampon applicator assembly comprising:
(a) an expandable tampon adapted to be placed in a vagina to absorb body fluid, said tampon having a first end portion and a second end portion;
(b) a withdrawal member attached to at least one of said first end portion and said second end portion of said tampon, said withdrawal member including a stopping member; and
(c) a single piece applicator having a leading end portion and a trailing end portion, said leading end portion engaging said tampon, and said trailing end portion adapted to releasably secure said stopping member against movement in at least one direction.

2. The assembly of claim 1, wherein said trailing end portion of said single piece applicator comprises a channel adapted to receive said withdrawal member.

3. The assembly of claim 1, wherein said single piece applicator has a generally planar shape.

4. The assembly of claim 1, wherein said single piece applicator has a generally cylindrical cross-section.

5. The assembly of claim 4, wherein said single piece applicator has a hollow interior through which said withdrawal member passes.

6. The assembly of claim 5, wherein said single piece applicator has a generally elliptical cross-section.

7. The assembly of claim 1, wherein said single piece applicator comprises at least one orientation indicator.

8. The assembly of claim 1, wherein said stopping member comprises a knot.

9. The assembly of claim 1, wherein said stopping member comprises a bead.

10. The assembly of claim 1, wherein said stopping member comprises a disk.

11. A tampon applicator assembly comprising:
(a) an expandable tampon adapted to be placed in a vagina and to absorb body fluid, said tampon having an expanded position and a contracted position; and
(b) a single piece applicator having a trailing end portion and a leading end portion, said leading end portion of said applicator engaging said tampon while said tampon is in its contracted position.

12. The assembly of claim 11, wherein said leading end portion is adapted to secure said tampon in its contracted position.

13. The assembly of claim 11, further comprising a withdrawal member attached with said tampon.

14. The assembly of claim 13, wherein said withdrawal member includes a stopping member.

15. The assembly of claim 14, wherein said trailing end portion is adapted to secure said stopping member against movement in at least one direction.

16. The assembly of claim 11, further comprising a dissolvable band around at least a portion of said tampon, said band adapted to hold said tampon in its contracted position.

17. The assembly of claim 1, wherein said single piece applicator comprises at least one orientation indicator.

18. A method of using a tampon within a vagina, comprising:
(a) inserting a tampon applicator assembly into a vagina, said assembly including
(i) an expandable tampon adapted to absorb body fluid, said tampon having a first end portion and a second end portion;
(ii) a withdrawal member attached to at least one of said first end portion and said second end portion of said tampon, said withdrawal member including a stopping member; and
(iii) a single piece applicator having a leading end portion and a trailing end portion, said leading end portion engaging said tampon, and said trailing end portion adapted to releasably secure said stopping member against movement in at least one direction;
(b) releasing said tampon from said single piece applicator; and
(c) withdrawing said single piece applicator from the vagina.

19. The method of claim 18, wherein said assembly further comprises a withdrawal member having a first and second end portion, said first end portion attached with said tampon and second end portion releasably secured against movement in at least one direction by said applicator; and
wherein releasing said tampon includes releasing said withdrawal member from said applicator.

20. The method of claim 18, wherein said assembly further comprises a dissolvable band positioned around at least a portion of said tampon to releasably hold said tampon against expansion; and wherein releasing said tampon includes dissolving said band.
